(19) United States
(12) Patent Application Publication
(10) Pub. No.: US 2025/0014692 A1
(43) Pub. Date: Jan. 9, 2025

(54) METHOD, SYSTEM AND COMPUTER PROGRAM FOR MANAGING BIOMETRIC DATA THROUGH LINKAGE BETWEEN PATCH-TYPE BIO SIGNAL MEASURING DEVICE AND SMART WATCH

(71) Applicant: Wellysis Corp., Seoul (KR)

(72) Inventors: Young Juhn, Seoul (KR); Rick Hongryul Kim, Seoul (KR); Jong Woo Kim, Seoul (KR); Jung Soo Kim, Seoul (KR)

(73) Assignee: Wellysis Corp., Seoul (KR)

(21) Appl. No.: 18/382,197

(22) Filed: Oct. 20, 2023

(30) Foreign Application Priority Data
Jul. 4, 2023 (KR) 10-2023-0086291

(51) Int. Cl. G16H 10/60; A61B 5/00; H04M 1/72409; H04M 1/72412

(52) U.S. Cl. CPC G16H 10/60; A61B 5/002; A61B 5/6833; H04M 1/724095; H04M 1/72412; A61B 5/0022

(57) ABSTRACT

A biometric data management method includes: measuring a bio signal of a user by a patch-type bio signal measuring device; checking a first residual quantity of a battery of a smart watch; connecting wireless communication between the patch-type bio signal measuring device and the smart watch, and receiving, by the smart watch, first biometric data corresponding to the bio signal measured by the patch-type bio signal measuring device from the patch-type bio signal measuring device; transmitting, by the smart watch, the first biometric data received from the patch-type bio signal measuring device to a server; disconnecting the wireless communication between the patch-type bio signal measuring device and the smart watch, and entering the smart watch into a standby mode; checking, by the smart watch, a second residual quantity of the battery; and transmitting, by the smart watch or the server, information on requested biometric data to a candidate device.

### BACKGROUND OF THE INVENTION

### Field of the Invention

The present disclosure relates to a technique for managing biometric data of a user through linkage between a patch-type bio signal measuring device and a smart watch.

### Description of the Related Art

A patch-type bio signal measuring device is a device that measures a bio signal such as an electrocardiogram (ECG) by forming contact points between an electrode patch and various body parts (e.g., chest, wrist, ankle, etc.) of a user.

A user may easily measure the bio signal in daily life by attaching the patch-type bio signal measuring device to the body.

In addition, medical personnel may predict or diagnose the occurrence of heart diseases such as arrhythmia and cardiac arrest by receiving and analyzing bio signals measured through a patch-type bio signal measuring device.

A conventional patch-type bio signal measuring device transmits measured biometric data to a mobile terminal such as a smartphone of a user through short-range communication, and the mobile terminal transmits biometric data to a server using cellular communication, WI-FI communication, etc.

However, since a smartphone is not always worn on the body unlike a smart watch, there are many limitations in integrating and utilizing data measured on a smartphone with data measured on a patch-type bio signal measuring device. For example, when determining whether a user falls down using an accelerometer of a smartphone, it is difficult to determine whether the user drops the smartphone or fell over using the accelerometer of the smartphone.

In addition, since a usable time of the smartphone is shorter than that of the patch-type bio signal measuring device due to the limitation of battery capacity of the smartphone, a situation in which the patch-type bio signal measuring device operates by being powered on but does not operate due to the power-off of the smartphone, a situation in which a communication connection between two devices is impossible due to the insufficient battery capacity of the smartphone, and the like, may occur. When theses situations occur, there may be a missing part in which some of biometric data measured by the patch-type bio signal measuring device is missing, so a problem of lowering reliability and accuracy of the biometric data may occur.

### SUMMARY OF THE INVENTION

The present disclosure provides a method of improving reliability and accuracy of biometric data through linkage between a patch-type bio signal measuring device and a smart watch.

The present disclosure provides a method of improving reliability and accuracy of biometric data through linkage between a patch-type bio signal measuring device and a smart watch.

### Means for Solving the Problems

According to another embodiment of the present disclosure, a biometric data management method of a system includes: measuring a bio signal of a user by a patch-type bio signal measuring device: checking a first residual quantity of a battery of a smart watch: when it is determined that the first residual amount of the battery is sufficient, connecting wireless communication between the patch-type bio signal measuring device and the smart watch, and receiving, by the smart watch, first biometric data corresponding to the bio signal measured by the patch-type bio signal measuring device for a first time from the patch-type bio signal measuring device: transmitting, by the smart watch, the first biometric data received from the patch-type bio signal measuring device to a server: disconnecting the wireless communication between the patch-type bio signal measuring device and the smart watch, and entering the smart watch into a standby mode for a predetermined data transmission cycle: after the predetermined data transmission cycle, checking, by the smart watch, a second residual quantity of the battery; and when it is determined that the second residual quantity of the battery is not sufficient, transmitting, by the smart watch or the server, information on requested biometric data to a candidate device.

The biometric data management method may further include: connecting wireless communication between the patch-type bio signal measuring device and the candidate device, and receiving, by the candidate device, second biometric data corresponding to the bio signal measured by the patch-type bio signal measuring device for a second time based on information on the requested biometric data from the patch-type bio signal measuring device.

The smart watch may select the candidate device from a list of the candidate devices provided from the server.

The information on the requested biometric data may include a packet number.

The second biometric data may be generated from a second bio signal subsequent to a first bio signal corresponding to the first biometric data.

The biometric data management method may further include: re-connecting the wireless communication between the patch-type bio signal measuring device and the smart watch when it is determined that the second residual quantity of the battery is sufficient; and after the re-connecting, receiving, by the smart watch, second biometric data corresponding to the bio signal measured by the patch-type bio signal measuring device for a second time from the patch-type bio signal measuring device.

The biometric data management method may further include: setting the data transmission cycle; and connecting the wireless communication between the patch-type bio signal measuring device and the smart watch, and requesting, by the smart watch, the patch-type bio signal measuring device to start measuring the bio signal.

The setting of the data transmission cycle may be variably determined according to at least one of a type of the bio signal, a communication environment, and a battery status.

The server may analyze the received biometric data and check whether there is missing biometric data.

The patch-type bio signal measuring device may be a patch-type device detachable from a body of a user, and the smart watch may be a wearable device worn on a wrist of the user.

According to another embodiment of the present disclosure, a biometric data management system includes: a patch-type bio signal measuring device configured to measure a bio signal of a user: a smart watch configured to receive biometric data corresponding to the bio signal measured by the patch-type bio signal measuring device from the patch-type bio signal measuring device; and a server configured to receive the biometric data from the smart watch, in which a first residual quantity of a battery of the smart watch is checked, and when it is determined that the first residual amount of the battery is sufficient, wireless communication is connected between the patch-type bio signal measuring device and the smart watch, and the smart watch receives first biometric data corresponding to the bio signal measured by the patch-type bio signal measuring device for a first time from the patch-type bio signal measuring device, the wireless communication is disconnected between the patch-type bio signal measuring device and the smart watch, and the smart watch enters a standby mode for a predetermined data transmission cycle, after the predetermined data transmission cycle, the smart watch checks a second residual quantity of the battery; and when it is determined that the second residual quantity of the battery is not sufficient, the smart watch or the server transmits information on requested biometric data to a candidate device.

The biometric data management system may further include: wireless communication is connected between the patch-type bio signal measuring device and the candidate device, and the candidate device receives second biometric data corresponding to the bio signal measured by the patch-type bio signal measuring device for a second time based on information on the requested biometric data from the patch-type bio signal measuring device.

The smart watch may select the candidate device from a list of the candidate devices provided from the server.

The information on the requested biometric data information may include a packet number.

The second biometric data may be generated from a second bio signal subsequent to a first bio signal corresponding to the first biometric data.

When it is determined that the second residual amount of the battery is sufficient, wireless communication may be re-connected between the patch-type bio signal measuring device and the smart watch, and after the re-connecting, the smart watch may receive second biometric data corresponding to a bio signal measured by the patch-type bio signal measuring device for a second time from the patch-type bio signal measuring device.

The data transmission cycle may be set, the wireless communication may be connected to the patch-type bio signal measuring device, and the smart watch may request the patch-type bio signal measuring device to start measuring a bio signal.

The data transmission cycle may be variably determined according to at least one of a type of the bio signal, a communication environment, and a battery status.

The server may analyze the received biometric data and check whether there is missing biometric data.

According to another embodiment of the present disclosure for achieving the above-described object, a computer readable recording medium may store a program for executing the biometric data management method described above.

According to another embodiment of the present disclosure for achieving the above-described object, a program stored in a computer readable recording medium may include a program code for executing a biometric data management method.

### BRIEF DESCRIPTION OF THE DRAWING FIGURES

FIG. 1 is a conceptual diagram illustrating a biometric data management system according to an embodiment of the present disclosure.

FIG. 2 is a block diagram illustrating a configuration of a patch-type bio signal measuring device according to an embodiment of the present disclosure.

FIG. 3 is a block diagram illustrating a configuration of a smart watch according to an embodiment of the present disclosure.

FIG. 4 is a block diagram illustrating a configuration of a biometric data management server according to an embodiment of the present disclosure.

FIG. 5 is a flowchart illustrating an initial device registration process according to an embodiment of the present disclosure.

FIGS. 6A and 6B are conceptual diagrams illustrating an initial device registration process according to an embodiment of the present disclosure.

FIG. 7 is a timing diagram illustrating a biometric data management method of a biometric data management system when a residual quantity of a battery of a smart watch according to an embodiment of the present disclosure is sufficient.

FIG. 8 is a conceptual diagram illustrating biometric data according to an embodiment of the present disclosure.

FIG. 9 is a timing diagram illustrating a biometric data management method of a biometric data management system when a residual quantity of a battery of a smart watch according to an embodiment of the present disclosure is not sufficient.

FIG. 10 is a timing diagram illustrating an operation of a biometric data management server according to an embodiment of the present disclosure.

### DETAILED DESCRIPTION OF THE EXEMPLARY EMBODIMENTS

The following description illustrates only a principle of the present disclosure. Therefore, those skilled in the art may implement the principle of the present disclosure and invent various devices included in the spirit and scope of the present disclosure although not clearly described or shown in the present specification. In addition, it is to be understood that all conditional terms and exemplary embodiments mentioned in the present specification are obviously intended only to allow those skilled in the art to understand a concept of the present disclosure in principle, and the present disclosure is not limited to exemplary embodiments and states particularly mentioned as such.

The above-mentioned objects, features, and advantages will become more obvious from the following detailed description provided in relation to the accompanying drawings. Therefore, those skilled in the art to which the present disclosure pertains may easily practice a technical idea of the present disclosure.

Further, in describing the present disclosure, in the case in which it is judged that a detailed description of a well-known technology associated with the present disclosure may unnecessarily make the gist of the present disclosure unclear, it will be omitted. Hereinafter, embodiments of the present disclosure will be described in detail with reference to the accompanying drawings.

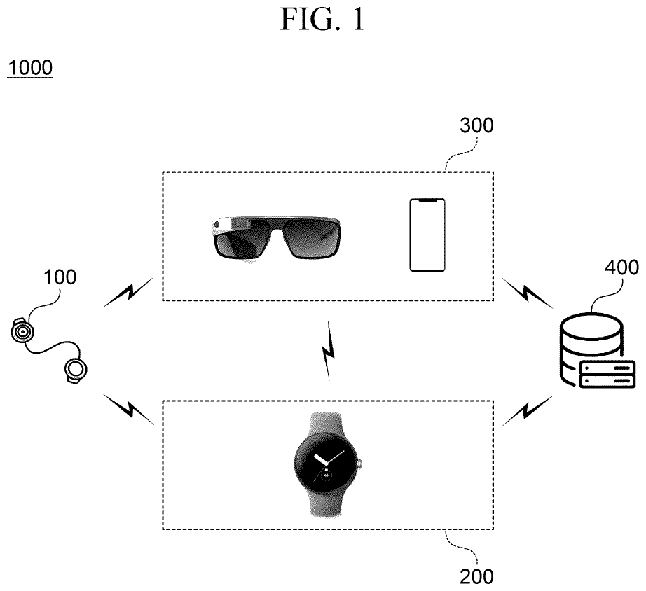

FIG. 1 is a conceptual diagram illustrating a biometric data management system according to an embodiment of the present disclosure.

Referring to FIG. 1, a biometric data management system 1000 may include part or all of a patch-type bio signal measuring device 100, a smart watch 200, a candidate device 300, and a biometric data management server 400.

The patch-type bio signal measuring device 100 is a patch-type bio signal measuring device detachable from a body of a user (subject to be measured), and may measure various physiological parameters of the user by being attached to the body of the user.

In the present specification, a bio signal measuring device implemented in a patch type has been described as an example, but this is only one implementation example of the present disclosure, and the bio signal measuring device 100 may be implemented in other types.

The smart watch 200 is a device that can be worn on a wrist of a user, and may be wirelessly connected to the patch-type bio signal measuring device 100 to receive biometric data measured by the patch-type bio signal measuring device 100 from the patch-type bio signal measuring device 100. In addition, the smart watch 200 may be worn on the wrist of the user to measure the bio signal of the user.

The candidate device 300 is a device that a user may carry, possess, wear, or the like, and may be implemented as smart glasses, a smart phone, or another smart watch separate from the smart watch 200. The candidate device 300 may replace the smart watch 200 in a situation where it is difficult for the smart watch 200 to operate according to the battery status of the smart watch 200 or the like, and receive the biometric data measured by the patch-type bio signal measuring device 100 from the patch-type bio signal measuring device 100.

The biometric data management server 400 may receive and store the biometric data from the smart watch 200 and/or the candidate device 300 and analyze the biometric data to diagnose a user's health condition or the like.

Meanwhile, the biometric data management system 100 including the above-described components 100, 200, 300, and 400 may serve to measure the user's bio signal and manage biometric data corresponding to the measured bio signal of the subject.

Specifically, a first residual quantity of a battery of the smart watch 200 is checked, and when it is determined that the first residual amount of the battery is sufficient, wireless communication is connected between the patch-type bio signal measuring device 100 and the smart watch 200, and the smart watch 200 may receive first biometric data corresponding to a bio signal measured by the patch-type bio signal measuring device 100 for a first time from the patch-type bio signal measuring device 100.

When the reception of the first biometric data is completed, the wireless communication may be disconnected between the patch-type bio signal measuring device 100 and the smart watch 200, and the smart watch 200 may enter a standby mode for a predetermined data transmission cycle.

After the predetermined data transmission cycle, the smart watch 200 checks the second residual quantity of the battery, and when it is determined that the second residual quantity of the battery is not sufficient, the smart watch 200 or the server 400 may transmit information (or information on the missing biometric data) on requested biometric data to the candidate device 300. Accordingly, when the wireless communication is connected between the candidate device 300 and the patch-type bio signal measuring device 100, the patch-type bio signal measuring device 100 may request and receive the biometric data corresponding to the patch-type bio signal measuring device 100 based on the information on the requested biometric data.

Meanwhile, the smart watch 200 and the candidate device 300 may transmit the biometric data received from the patch-type bio signal measuring device 100 to the biometric data management server 400 in real time or at a preset cycle.

Components constituting the biometric data management system 1000 of the present disclosure will be described below in more detail with reference to the accompanying drawings.

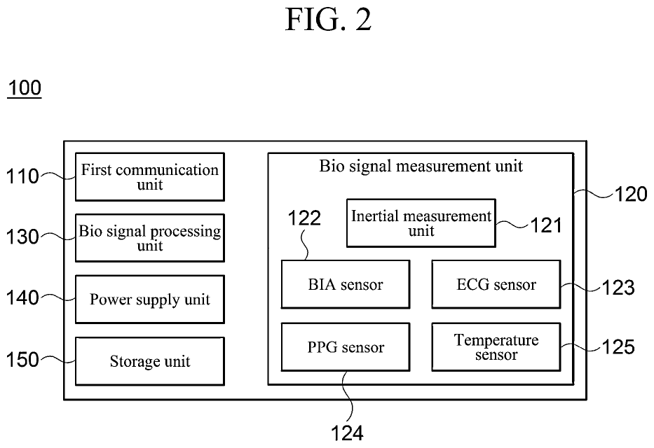

FIG. 2 is a block diagram illustrating a configuration of a patch-type bio signal measuring device according to an embodiment of the present disclosure. Referring to FIG. 2, the patch-type bio signal measuring device 100 may include part or all of a first communication unit 110, a bio signal measurement unit 120, a bio signal processing unit 130, a power supply unit 140, and a storage unit 150.

Here, the patch-type bio signal measuring device 100 is a device that measures bio signals such as electrocardiogram, electromyogram, body temperature, posture, and body fat by attaching electrode patches to various body parts (chest, wrist, ankle, etc.) of a user, and may refer to a device implemented in a patch type.

The first communication unit 110 may perform a communication function for the patch-type bio signal measuring device 100. For example, the first communication unit 110 may be implemented as a module that may perform a short-range wireless communication near field communication (NFC) communication, beacon communication, Bluetooth communication, Zigbee communication, WI-FI communication, Z-wave communication, and may be further implemented as a configuration that may perform LTE communication, 5G communication, radio-frequency identification (RFID) communication, and the like, that are long-range wireless communication.

Preferably, the first communication unit 110 may be implemented as a Bluetooth low energy (BLE) communication module.

The first communication unit 110 may establish a wireless communication connection with the smart watch 200 and/or the candidate device 300, and when the wireless communication is connected, may transmit and receive various biometric data and various data such as control commands to and from the smart watch 200 and/or the candidate device 300.

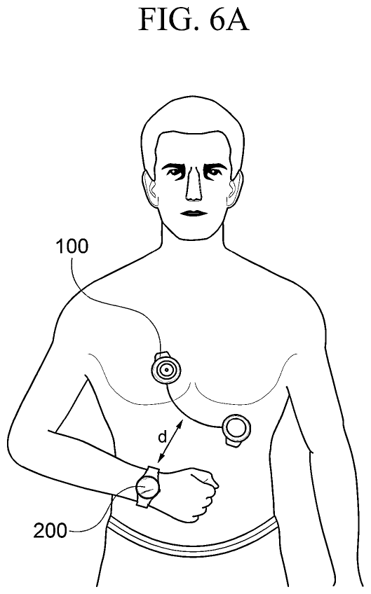

In the case of connecting first wireless communication between the patch-type bio signal measuring device 100 and the smart watch 200 and/or the candidate device 300 (that is, when the smart watch 200 and/or the candidate device 300 are not registered as communication capable devices in the patch-type bio signal measuring device 100), a device authentication procedure may be performed by performing an initial device registration process according to an embodiment of the present disclosure. An initial device registration process according to an embodiment of the present disclosure will be described later with reference to FIG. 5.

Meanwhile, when the wireless communication is connected with the smart watch 200 and/or the candidate device 300, the first communication unit 110 may receive a bio signal measurement start request, a bio signal measurement end request, a biometric data initialization request, or the like from the smart watch 200 and/or candidate device 300. When the bio signal measurement start request is received, the first communication unit 110 may transmit the biometric data measured for a preset period and stored in the storage unit 150 to the smart watch 200 and/or the candidate device 300.

The bio signal measurement unit 120 is composed of various sensors and may measure various bio signals such as brain waves, electrocardiogram, electromyogram, oxygen saturation, body temperature, body fat, and posture of a user.

As an example, the bio signal measurement unit 120 may include part or all of an inertial measurement unit 121, a bioelectrical impedance analysis (BIA) sensor 122, an electrocardiograph (ECG) sensor 123, a photoplethysmography (PPG) sensor 124, and a temperature sensor 125. Although not illustrated in FIG. 2, the bio signal measurement unit 120 may further include part or all of a galvanic skin response sensing module, an oxygen saturation (spO2) sensing module, a body fat sensing module, and an electromyography (EMG) sensing module.

The bio signal processing unit 130 may generate biometric data corresponding to the bio signal in the bio signal measurement unit 120. Specifically, a data format of a live packet generated from the bio signal processing unit 130 may be as shown in Table 1 below.

TABLE 1

| | | | | | | |
|---|---|---|---|---|---|---|
| Byte | 4 | 2 | 384 | 1 | 1 | 1 |
| Role | Packet | ECG Data | ECG Data | CRC | ETX | Status |
| | Number | Length | | | | |

Table 1 above is the format of the live packet, and the live packet according to an embodiment of the present disclosure may be implemented with 393 bytes. “Packet Number” refers to the packet number of biometric data, and may increase by 1 every second and start from 0. “ECG Data Length” may refer to a length of ECG data, “ECG data” may refer to ECG Data, “CRC” may refer to information for determining the validity of ECG data, “ETX” may refer to information for determining the validity of a live packet structure, and “Status” may refer to a status of the live packet. Here, “Status” may be as shown in Table 2.

TABLE 2

| Hex | 00 | 01 | 02 | 04 | 08 | 10 | 20 | 40 | 80 |
|---|---|---|---|---|---|---|---|---|---|
| Status | Default | Live | Lead On | Memory | Switch | Memory | Boot | IMU | Symptom |
| | | | | Write | | Read/ | Flag | Fall | |
| | | | | Recover | | Write | | | |
| | | | | | | Fail | | | |

Table 2 above shows values indicating the status of the live packet.

In Table 2, “Default” may refer to the default value, “Live” may refer to a state in which no further data is written to the storage unit 150 and only live ECG is transmitted, “Lead On” may refer to a state where the patch-type bio signal measuring device 100 is attached to the body, “Memory Write Recover” may refer to a state in which the storage (writing) of the storage unit 150 of the patch-type bio signal measuring device 100 has failed and may be recovered and stored again, “Switch” means pressing a power button of the patch-type bio signal measuring device 100, Memory Read/Write Fail” may refers to failure to fetch data from or write data to the storage unit 150 of the patch-type bio signal measuring device 100, “Boot Flag” may refer to turning on the patch-type bio signal measuring device 100, “IMU Fall” may refer to fall detection, and “Symptom” may refer to symptom detection through the power button of the patch-type bio signal measuring device 100.

In addition, the bio signal processing unit 130 may further include additional information in the live packet to generate a record packet as illustrated in Table 3 below. A data format of a record packet generated from the bio signal processing unit 130 may be as shown in Table 3 below. The biometric data may be stored in the storage unit 150 according to an embodiment of the present disclosure in the format of the record packet.

TABLE 3

| | | | | | | | |
|---|---|---|---|---|---|---|---|
| Byte | 393 | 2 | 1 | 2 | 1 | 8 | 1 |
| Role | Live | IMU Fall | HR | Battery | Status | Mark or | BLE |
| | Packet | Detection | | | | BP/Reboot | Status |

Table 3 above is a table showing the record packet structure of biometric data. Referring to Table 3, a record packet can be implemented with 408 bytes. “Live Packet” may refer to the live packet of Table 1 above, “IMU Fall Detection” may refer to a fall detection value (ms), “HR” may refer to a heart rate, “Battery” may refer to a battery status of the patch-type bio signal measuring device 100, “Status” may refer to a status of a record packet as shown in Table 2 above, “Mark or BP/Reboot” may refer to the presence or absence of a Mark value input, and “BLE status” may refer to a status of BLE communication. Here, the battery status may include information on a residual quantity of a battery.

The storage unit 150 may store biometric data corresponding to a bio signal measured by the patch-type bio measurement patch 100.

The power supply unit 140 may include a battery, and supply power for the patch-type bio signal measuring device 100 to operate. Here, turning the power unit 140 on/off can be implemented by manipulating a button formed in one area of the patch-type bio signal measuring device 100, and the button may also be used as a button to set an operation mode of the patch-type bio signal measuring device 100.

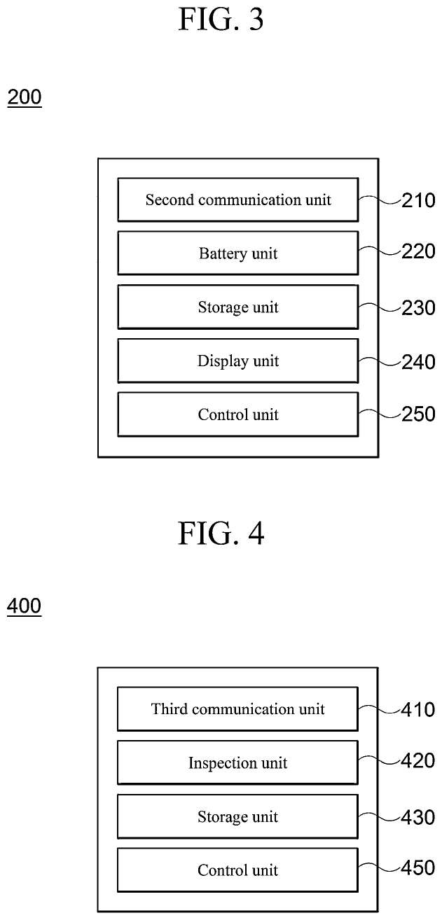

FIG. 3 is a block diagram illustrating a configuration of a smart watch according to an embodiment of the present disclosure. Referring to FIG. 3, the smart watch 200 may include a part or mode of a second communication unit 210, a battery unit 220, a storage unit 230, a display unit 240, and a control unit 250.

The second communication unit 210 may perform a communication function for the smart watch 200. Specifically, the second communication unit 210 may communicate with the patch-type bio signal measuring device 100, the candidate device 300, and the biometric data management server 400 to transmit and receive various data.

In particular, the second communication unit 210 may receive biometric data from the patch-type bio signal measuring device 100 and transmit the received biometric data to the biometric data management server 300. Additionally, the second communication unit 210 may transmit information on the requested biometric data to the candidate device 300.

As one implementation example, the second communication unit 210 may be implemented as a BLE communication module for communicating with the patch-type bio signal measuring device 100 and the candidate device 300, and a cellular or Wi-Fi communication module for communicating with the biometric data management server 400.

The battery unit 220 may supply power for the operation of the smart watch 200. Additionally, the battery unit 220 may provide the information on the residual quantity of the battery to the control unit 250.

The storage unit 230 may store various data necessary for the operation of the smart watch 200. Specifically, the storage unit 230 may store a list of candidate devices 300, biometric data received from the patch-type bio signal measuring device 100, etc. Here, the list of candidate devices 300 may be a list of devices that are registered as a device that may receive biometric data from the patch-type bio signal measuring device 100 on behalf of the smart watch 200 in the situation where the smart watch 200 may not operate.

The display unit 240 may output various data related to the smart watch 200 or display a user interface for user input. Specifically, the display unit 240 may display an interface including data such as received biometric data, biometric data transmission and reception status, and battery status.

In addition, the display unit 240 may display an interface for inputting user control commands, such as a request to start/end measuring bio signal, a request for biometric data, etc.

The control unit 250 controls a general operation of the smart watch 200. Specifically, the control unit 250 may control all or part of the second communication unit 210, the battery unit 220, the storage unit 230, and the display unit 240.

In particular, the control unit 250 may determine whether a battery is sufficient to transmit and receive biometric data from the patch-type bio signal measuring device 100 based on the residual quantity of the battery unit 220. In this case, the control unit 250 may determine whether a battery is sufficient by considering not only the residual quantity of the battery of the smart watch 200, but also past battery consumption patterns and a size (capacity) of biometric data.

When it is determined that the residual quantity of the battery unit 220 is sufficient, the control unit 250 may control the communication unit 210 to connect wireless communication between the patch-type bio signal measuring device 100 and the smart watch 200, and may control to receive first biometric data corresponding to the bio signal measured by the patch-type bio signal measuring device for a first time from the patch-type bio signal measuring device 100.

When the first biometric data is received, the control unit 250 may control to disconnect the wireless communication between the patch-type bio signal measuring device 100 and the smart watch 200, and control the smart watch 200 to enter a standby mode for a predetermined data transmission cycle.

After the predetermined data transmission cycle, the control unit 250 checks the residual quantity of the battery unit 220, and when it is determined that the residual quantity of the battery unit 220 is sufficient, the control unit 250 may control the communication unit 210 to connect the wireless communication between the patch-type bio signal measuring device 100 and the smart watch 200, and may control to receive second biometric data corresponding to the bio signal measured by the patch-type bio signal measuring device for a second time from the patch-type bio signal measuring device 100.

However, when it is determined that the residual quantity of the battery unit 220 is not sufficient, the control unit 250 may control a second communication unit 210 to select at least one candidate device from the list of candidate devices and transmit the information on the requested biometric data to the candidate device 300. In this case, the control unit 250 may determine the candidate device 300 according to the distance between the candidate device 300 and the patch-type bio signal measuring device 100, the residual quantity of the battery of the candidate device 300, the type of the candidate device 300, the distance between the candidate device 300 and the smart watch 200, and the like.

Meanwhile, the candidate devices 300 may be a device that may receive the biometric data from the patch-type bio signal measuring device 100 on behalf of the smart watch 200 in the situation where the smart watch 200 may not operate, and may be implemented to include all the configuration modules of the smart watch 200 described above. Therefore, a detailed description of the configuration modules of the candidate device 300 will be omitted.

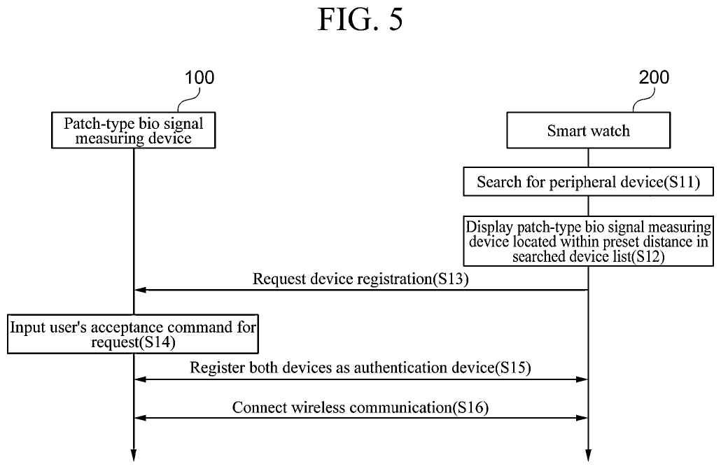

FIG. 4 is a block diagram illustrating a biometric data management server according to an embodiment of the present disclosure. Referring to FIG. 4, the biometric data management server 400 may include part or all of a third communication unit 410, an inspection unit 420, a storage unit 430, and a control unit 440.

The third communication unit 410 may communicate with the smart watch 200 and/or the candidate device 300 to transmit and receive biometric data, various control commands, and the like.

The inspection unit 420 may analyze whether the electrocardiogram, pulse, body temperature, electromyogram, etc., of the user are normal or abnormal based on the received biometric data, and generate inspection data according to the analysis. Here, inspection data may include inspection results and inspection progress information based on the analysis of the biometric data of the user.

Here, the inspection progress information may include whether additional biometric data is needed to generate inspection data, the type of additional biometric data required, inspection progress situation, etc.

The storage unit 430 may store various data required for the biometric data management server 400. Specifically, the storage unit 430 may store the list of smart watches 200 registered for authentication, the list of candidate devices 300 registered for authentication, the biometric data, the inspection data, etc. Here, the list of devices registered for authentication may be updated in real time.

The control unit 440 may control various operations of the biometric data management server 400. Specifically, the control unit 440 may control all or part of the third communication unit 410, the inspection unit 420, and the storage unit 430.

In particular, the control unit 440 may determine whether there is missing data in the received biometric data. As an example, the control unit 440 may detect missing biometric data by analyzing the packet number of the received biometric data.

In addition, the control unit 440 may transmit information on the requested biometric data including the packet number of the missing biometric data and a request command to the smart watch 200 or the candidate device 300, and the smart watch 200 or the candidate device 300 that has received the command may request and receive the missing biometric data from the patch-type bio signal measuring device 100.

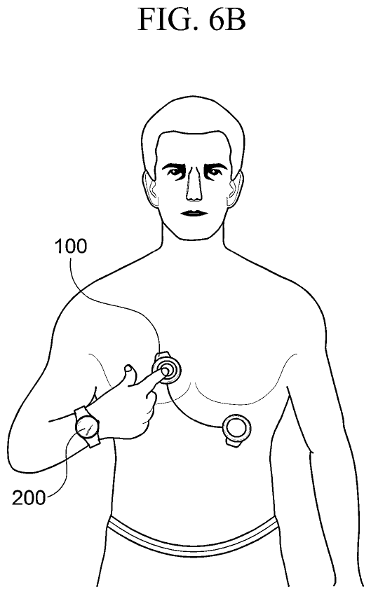

FIGS. 5 and 6 are diagrams illustrating an initial device registration process according to an embodiment of the present disclosure. Referring to FIG. 5, the control unit 250 of the smart watch 200 performs a search for peripheral devices (S11), and when the patch-type bio signal measuring device 100 and the smart watch 200 are located within a preset distance, the display unit 240 of the smart watch 200 may display the patch-type bio signal measuring device 100 in the list of searched devices (S12). Here, the preset distance may be within 1 meter.

As an example, as illustrated in FIG. 6A, when a user raises his/her hand while wearing the smart watch 200 and brings the smart watch 200 closer to the patch-type bio signal measuring device 100 within 1 meter (d), the display unit 240 of the smart watch 200 may display the patch-type bio signal measuring device 100 in the list of searched devices.

Then, the control unit 250 of the smart watch 200 requests the patch-type bio signal measuring device 100 to register the device (S13), and when the acceptance of the request is input from the user in the patch-type bio signal measuring device 100 (S14), the patch-type bio signal measuring device 100 and the smart watch 200 may register both devices as the authentication device (S15). The wireless communication may be connected between the patch-type bio signal measuring device 100 and the smart watch 200.

For example, as illustrated in FIG. 6B, the action of the user pressing the button of the patch-type bio signal measuring device 100 may correspond to the acceptance command input of the user for the device registration request.

Meanwhile, after the initial device registration process, the patch-type bio signal measuring device 100 and the smart watch 200 register their respective device information in the authentication device list, and in the future, the wireless communication may be automatically connected without the device registration process.

Meanwhile, the above-described initial device registration process may be equally applied to the candidate device 300.

The patch-type electrocardiogram measuring device 100, which is attached to the user's body and performs a Holter monitor function, generally performs cardiac monitoring for 24 to 72 hours. When the patch-type electrocardiogram measuring device 100 does not have a device registration process, a security problem may occur in which other users may access the patch-type ECG measurement device 100 of the user and receive the biometric data of the user. However, according to the present disclosure, by enabling transmission and reception of biometric data only between devices authenticated through an initial device registration process between a patch-type bio signal measuring device 100 and other devices (e.g., smart watch and candidate device), it is possible to solve a security problem of biometric data of a user.

In addition, the patch-type electrocardiogram measuring device 100 generally does not have a display means to minimize battery consumption, so the device registration process may be difficult, but the present disclosure proposes the initial device registration process for the patch-type bio signal measuring device 100, thereby enabling the device registration process even when there is no display means.

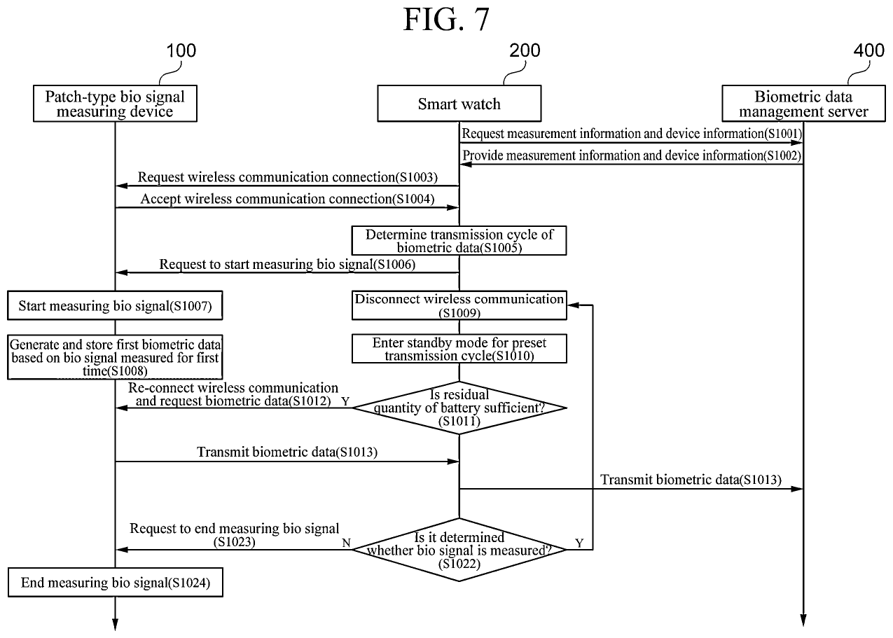

FIG. 7 is a timing diagram illustrating a biometric data management method of a biometric data management system 1000 when the residual quantity of the battery of the smart watch 200 is sufficient. Each step illustrated in FIG. 7 may be performed under the control of a control unit that controls operations of each device 100, 200, and 400.

Referring to FIG. 7, the smart watch 200 may request measurement information and device information from the biometric data management server 400 (S1001) and receive the measurement information and the device information (S1002). Here, the measurement information is information on a bio signal to be measured from the user and may include the type of bio signal to be measured, time, etc. Additionally, the device information may be a device authenticated as a device that can be connected to the patch-type bio signal measuring device 100 through the initial device registration process.

Then, the smart watch 200 may request a wireless communication connection from the patch-type bio signal measuring device 100 based on the device information (S1003), and the patch-type bio signal measuring device 100 may accept the wireless communication connection (S1004).

Then, when the wireless communication is connected between the patch-type bio signal measuring device 100 and the smart watch 200, the smart watch 200 may determine the transmission cycle of biometric data (S1005), and the smart watch 200 may request the patch-type bio signal measuring device 100 to start measuring the bio signal (S1006).

Here, the smart watch 200 may determine the transmission cycle of the biometric data according to the type of bio signal to be measured, the battery status of the smart watch 200, the battery status of the patch-type bio signal measuring device 100, the capacity of the biometric data, the sensitivity of the wireless communication, etc.

Then, the smart watch 200 may request the patch-type bio signal measuring device 100 to start measuring the bio signal (S1006). The request may include the transmission cycle of the biometric data determined in step S1005.

Meanwhile, the smart watch 200 may receive the preset transmission cycle from the biometric data management server 400 or the patch-type bio signal measuring device 100.

The patch-type bio signal measuring device 100 may measure the bio signal of the user according to a request to start measuring the bio signal from the smart watch 200 (S1007), and generate and store the biometric data corresponding to the measured bio signal (S1008).

Meanwhile, after requesting to start measuring the bio signal, the smart watch 200 may disconnect the wireless communication with the patch-type bio signal measuring device 100 (S1009) and enter the standby mode for the preset transmission cycle (S1010). Here, the standby mode is a mode to reduce the battery consumption of the smart watch 200 and may be a state in which the operation of the BLE communication module with the patch-type bio signal measuring device 100 may be turned off and the communication with the patch-type bio signal measuring device 100 may be disconnected.

And, after the predetermined transmission cycle, the smart watch 200 may check whether the residual quantity of the battery is sufficient to transmit and receive the biometric data from the patch-type bio signal measuring device 100 (S1011). In this case, the smart watch 200 may determine whether the residual quantity of the battery is sufficient by considering not only the residual quantity of the current battery of the smart watch 200, but also the current battery consumption level, the past battery consumption pattern, the capacity of the biometric data, etc.

When it is determined that the residual quantity of the battery of the smart watch 200 is sufficient (S1011: Y), the smart watch 200 may request re-connection of the wireless communication from the patch-type bio signal measuring device 100, request the biometric data from the patch-type bio signal measuring device 100 (S1012). Additionally, the patch-type bio signal measuring device 100 may transmit the first biometric data generated by measuring the bio signal for the first time to the smart watch 200 (S1013).

Then, the smart watch 200 may determine whether to continue measuring the bio signal (S1022). When the measurement of the bio signal does not continue (S1022: N), the smart watch 200 may request the patch-type bio signal measuring device 100 to end measuring the bio signal (S1023), and the patch-type bio signal measuring device 100 may end measuring the bio signal (S1024).

When the measurement of the bio signal continues (S1022: Y), the smart watch 200 may disconnect the wireless communication with the bio signal measuring device 100 again (S1009) and enter standby mode for the predetermined transmission cycle (S1010). And, after the predetermined transmission cycle, the smart watch 200 may check whether the residual quantity of the battery is sufficient to transmit and receive the biometric data from the patch-type bio signal measuring device 100 (S1011).

When it is determined that the residual quantity of the battery of the smart watch 200 is sufficient (S1011: Y), the smart watch 200 may request re-connection of the wireless communication from the patch-type bio signal measuring device 100, request the second biometric data measured for the second time from the patch-type bio signal measuring device 100 (S1012). Then, the patch-type bio signal measuring device 100 may transmit the second biometric data generated by measuring the bio signal for the second time to the smart watch 200 (S1013).

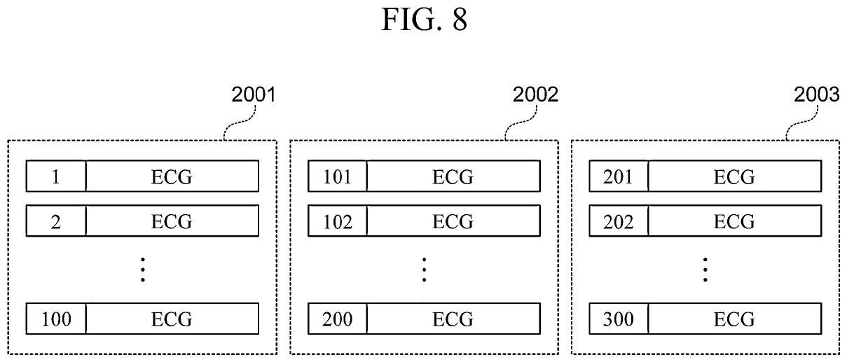

Here, the second biometric data may be data generated from a second bio signal subsequent to the first bio signal corresponding to the first biometric data. Meanwhile, the smart watch 200 may transmit the received biometric data to the biometric data management server 400 in real time or in aggregate (S1014). As described above, the patch-type bio signal measuring device 100 may perform a Holter monitor function, generally measure a bio signal continuously for 24 to 72 hours, and store biometric data corresponding to bio signals measured at predetermined times. In this case, the predetermined time may correspond to the transmission cycle. This will be described in more detail with reference to FIG. 8.

FIG. 8 is a conceptual diagram illustrating biometric data according to an embodiment of the present disclosure.

Referring to FIG. 8, the patch-type bio signal measuring device 100 can continuously measure bio signals. For example, a data packet may have an increasing packet number in chronological order, biometric data measured for a predetermined period from a time of a first request from the smart watch 200 may be the same as 2001, biometric data measured for a predetermined period from a time of a second request by the smart watch 200 may be the same as 2002, and biometric data measured for a predetermined period from a time of a third request from the smart watch 200 may be the same as 2003.

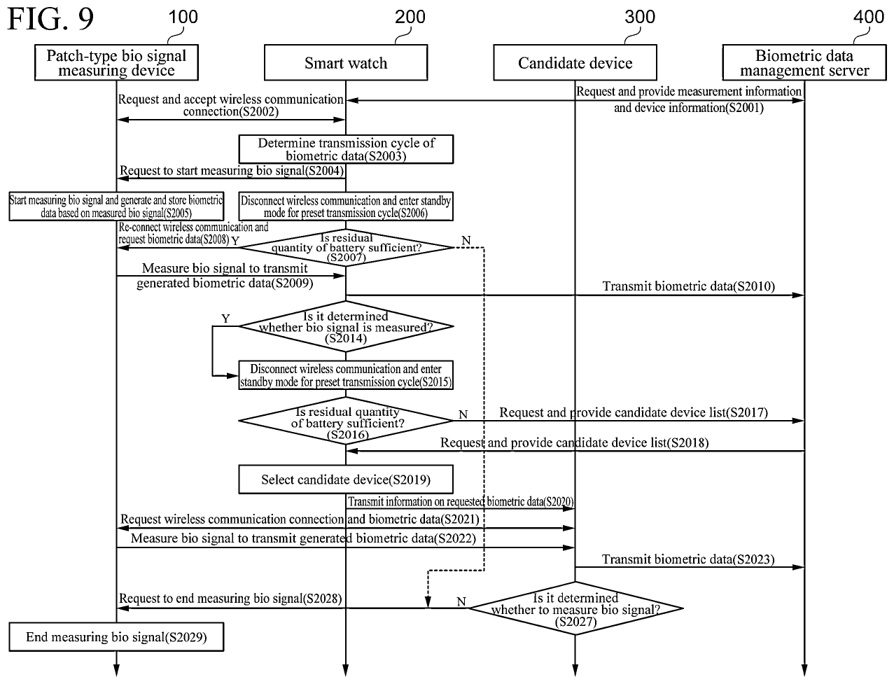

Meanwhile, when it is determined that the battery residual quantity of the smart watch 200 is insufficient, the smart watch 200 may designate a candidate device 300 receiving biometric data on behalf of the smart watch 200, and delegate the authority to receive the biometric data to the corresponding candidate device 300. This will be described below with reference to FIG. 9.

FIG. 9 is a timing diagram illustrating a biometric data management method of a biometric data management system when a residual quantity of a battery of a smart watch according to an embodiment of the present disclosure is not sufficient. In describing FIG. 9, description of steps of the same function as in FIG. 7 will be omitted. Each step illustrated in FIG. 9 may be performed under the control of a control unit that controls operations of each device 100, 200, 300, and 400.

In FIG. 9, when it is determined that the battery is not sufficient (S2016: N), the smart watch 200 may request a list of candidate devices 300 from the biometric data management server 400 (S2017) and receive a list of candidate devices. (S2018).

Then, the smart watch 200 may select the candidate device 300 based on the list of candidate devices 300 (S2019).

Then, after the smart watch 200 wirelessly communicates with the selected candidate device 300, the smart watch 200 may transmit the information on the requested biometric data to the candidate device 300 (S2020). Here, the candidate device 300 may be a device that has been authenticated and registered according to the initial device registration process described above.

In addition, the candidate device 300 may wirelessly communicate with the patch-type signal measurement device 100 and request transmission of missing biometric data from the patch-type signal measurement device 100 based on information on the requested biometric data (S2021). Here, the information on the requested biometric data may include the packet number of the requested biometric data.

The patch-type bio signal measuring device 100 may detect biometric data matching the packet number among the biometric data stored in the storage unit 150 based on the packet number included in the information on the requested biometric data. The patch-type bio signal measuring device 100 may transmit the detected biometric data to the candidate device 300 (S2022).

As an example, when only the first biometric data corresponding to the first bio signal measured for the first time is transmitted to the smart watch 200, the requested biometric data may be the second biometric data corresponding to the second biometric data measured for the second time subsequent to the first time. In this case, the patch-type bio signal measuring device 100 may detect the second biometric data stored in the storage unit 150 and transmit the second biometric data to the candidate device 300.

Meanwhile, the candidate device 300 may transmit the received biometric data to the biometric data management server 400 in real time or in aggregate (S2023).

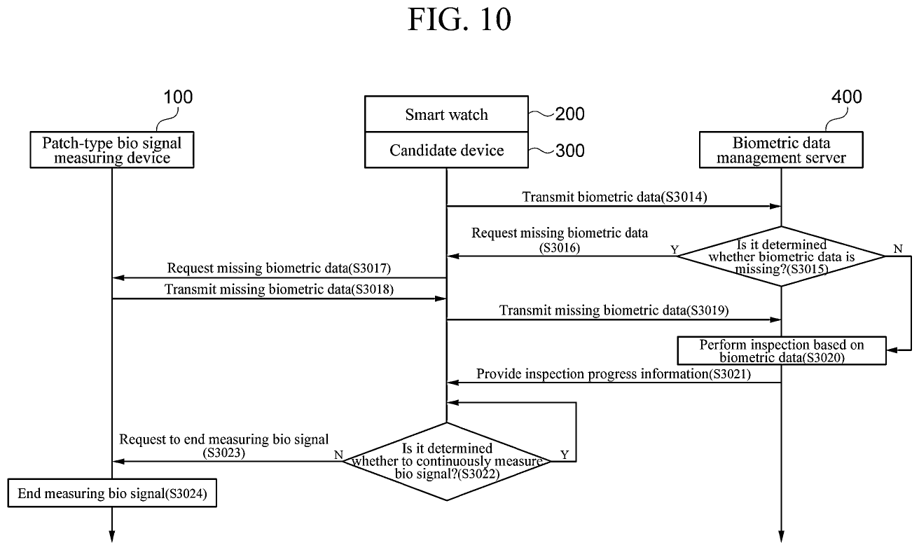

FIG. 10 is a timing diagram illustrating an operation of a biometric data management server according to an embodiment of the present disclosure. Referring to FIG. 10, the biometric data management server 400 may receive the biometric data from the smart watch 200 and/or the candidate device 300 (S3014), and analyze the received biometric data to determine whether the biometric data is missing (S3015). As an example, the biometric data management server 400 may determine the missing biometric data by analyzing the packet number of the received biometric data.

If there is no missing of biometric data (S3015: N), the biometric data management server 400 may perform an inspection based on the biometric data (S3020).

Then, the biometric data management server 400 may transmit inspection progress information to the smart watch 200 and/or the candidate device 300 (S3021), and the smart watch 200 and/or the candidate device 300 may determine whether to proceed with bio signal measurement based on the inspection progress information (S3022).

When it is determined that the measurement of the bio signal does not proceed (S3022: N), the smart watch 200 and/or candidate device 300 may request the patch-type bio signal measuring device 100 to end measuring the bio signal (S2023), and the patch-type bio signal measuring device 100 may end measuring the bio signal (S3024).

When the biometric data is missing (S3015: Y), the biometric data management server 400 may request the missing biometric data from the smart watch 200 and/or the candidate device 300 (S3016).

Then, the smart watch 200 and/or the candidate device 300 requests the missing biometric data from the patch-type bio signal measuring device 100 (S3017) and receives the missing biometric data (S3018), and the smart watch (200) and/or the candidate device 300 may transmit the missing biometric data to the biometric data management server 400 (S3019).

Meanwhile, the biometric data management server 400 may perform an examination of a body of a user based on the received biometric data, and monitor the body of the user or determine abnormal symptoms based on the biometric data.

According to various embodiments of the present disclosure, a process of connecting wireless communication between a smart watch (or candidate device) and a patch-type bio signal measuring device and disconnecting wireless communication when biometric data is received is repeated according to a predetermined data transmission cycle, thereby efficiently using batteries of the devices.

In addition, according to various embodiments of the present disclosure, when a battery of a smart watch is not sufficient, biometric data is received from a patch-type bio signal measuring device through a candidate device to make sure that there is no missing part of the biometric data, thereby increasing reliability and accuracy of the biometric data.

Hereinabove, the present disclosure has been described with reference to exemplary embodiments. All exemplary embodiments and conditional illustrations disclosed in the present disclosure have been described to intend to assist in the understanding of the principle and the concept of the present disclosure by those skilled in the art to which the present disclosure pertains. Therefore, it will be understood by those skilled in the art to which the present disclosure pertains that the present disclosure may be implemented in modified forms without departing from the spirit and scope of the present disclosure.

Therefore, exemplary embodiments disclosed herein should be considered in an illustrative aspect rather than a restrictive aspect. The scope of the present disclosure should be defined by the claims rather than the above-mentioned description, and equivalents to the claims should be interpreted to fall within the present disclosure.

Meanwhile, the above-described driving assistance guidance providing method according to various embodiments of the present disclosure may be implemented as a program and provided to servers or devices. Therefore, the respective apparatuses may access the server or the device in which the program is stored to download the program.

In addition, the methods according to various exemplary embodiments of the present disclosure described above may be implemented as programs and be provided in a state in which it is stored in various non-transitory computer-readable media. The non-transitory computer-readable medium is not a medium that stores data therein for a while, such as a register, a cache, a memory, or the like, but means a medium that semi-permanently stores data therein and is readable by an apparatus. In detail, the various applications or programs described above may be stored and provided in the non-transitory computer readable media such as a compact disk (CD), a digital versatile disk (DVD), a hard disk, a Blu-ray disk, a universal serial bus (USB), a memory card, and a read only memory (ROM).

According to various embodiments of the present disclosure, by enabling transmission and reception of biometric data only between devices authenticated through an initial device registration process between a patch-type bio signal measuring device and other devices (e.g., smart watch and candidate device), it is possible to solve a security problem of biometric data of a user.

In addition, according to various embodiments of the present disclosure, a process of connecting wireless communication between a smart watch (or candidate device) and a patch-type bio signal measuring device and disconnecting wireless communication when biometric data is received is repeated according to a predetermined data transmission cycle, thereby efficiently using batteries of the devices.

In addition, according to various embodiments of the present disclosure, when a battery of a smart watch is not sufficient, biometric data is received from a patch-type bio signal measuring device through a candidate device to make sure that there is no missing part of the biometric data, thereby increasing reliability and accuracy of the biometric data.

Although the exemplary embodiments of the present disclosure have been illustrated and described hereinabove, the present disclosure is not limited to the specific exemplary embodiments described above, but may be variously modified by those skilled in the art to which the present disclosure pertains without departing from the scope and spirit of the disclosure as claimed in the claims. These modifications should also be understood to fall within the technical spirit and scope of the present disclosure.

What is claimed is:

1. A biometric data management method of a system, comprising:
measuring a bio signal of a user by a patch-type bio signal measuring device;
checking a first residual quantity of a battery of a smart watch;
when it is determined that the first residual amount of the battery is sufficient, connecting wireless communication between the patch-type bio signal measuring device and the smart watch, and receiving, by the smart watch, first biometric data corresponding to the bio signal measured by the patch-type bio signal measuring device for a first time from the patch-type bio signal measuring device;
transmitting, by the smart watch, the first biometric data received from the patch-type bio signal measuring device to a server;
disconnecting the wireless communication between the patch-type bio signal measuring device and the smart watch, and entering the smart watch into a standby mode for a predetermined data transmission cycle;
after the predetermined data transmission cycle, checking, by the smart watch, a second residual quantity of the battery; and
when it is determined that the second residual quantity of the battery is not sufficient, transmitting, by the smart watch or the server, information on requested biometric data to a candidate device.

2. The biometric data management method of claim 1, further comprising:
connecting wireless communication between the patch-type bio signal measuring device and the candidate device, and receiving, by the candidate device, second biometric data corresponding to the bio signal measured by the patch-type bio signal measuring device for a second time based on information on the requested biometric data from the patch-type bio signal measuring device.

3. The biometric data management method of claim 1, wherein the smart watch selects the candidate device from a list of the candidate devices provided from the server.

4. The biometric data management method of claim 1, wherein the information on the requested biometric data includes a packet number.

5. The biometric data management method of claim 2, wherein the second biometric data is generated from a second bio signal subsequent to a first bio signal corresponding to the first biometric data.

6. The biometric data management method of claim 1, further comprising:
re-connecting the wireless communication between the patch-type bio signal measuring device and the smart watch when it is determined that the second residual quantity of the battery is sufficient; and
after the re-connecting, receiving, by the smart watch, second biometric data corresponding to the bio signal measured by the patch-type bio signal measuring device for a second time from the patch-type bio signal measuring device.

7. The biometric data management method of claim 1, further comprising:
setting the data transmission cycle; and
connecting the wireless communication between the patch-type bio signal measuring device and the smart watch, and requesting, by the smart watch, the patch-type bio signal measuring device to start measuring the bio signal.

8. The biometric data management method of claim 7, wherein the setting of the data transmission cycle is variably determined according to at least one of a type of the bio signal, a communication environment, and a battery status.

9. The biometric data management method of claim 1, wherein the server analyzes the received biometric data and checks whether there is missing biometric data.

10. The biometric data management method of claim 1, wherein the patch-type bio signal measuring device is a patch-type device detachable from a body of the user, and
the smart watch is a wearable device worn on a wrist of the user.

11. A biometric data management system, comprising:
a patch-type bio signal measuring device configured to measure a bio signal of a user;
a smart watch configured to receive biometric data corresponding to the bio signal measured by the patch-type bio signal measuring device from the patch-type bio signal measuring device; and
a server configured to receive the biometric data from the smart watch,
wherein a first residual quantity of a battery of the smart watch is checked, and when it is determined that the first residual amount of the battery is sufficient, wireless communication is connected between the patch-type bio signal measuring device and the smart watch, and the smart watch receives first biometric data corresponding to a bio signal measured by the patch-type bio signal measuring device for a first time from the patch-type bio signal measuring device,
the wireless communication is connected between the patch-type bio signal measuring device and the smart watch, and the smart watch enters a standby mode for a predetermined data transmission cycle,
after the predetermined data transmission cycle, the smart watch checks a second residual quantity of the battery; and
when it is determined that the second residual quantity of the battery is not sufficient, the smart watch or the server transmits information on requested biometric data to a candidate device.

12. The biometric data management system of claim 11, further comprising:
wireless communication is connected between the patch-type bio signal measuring device and the candidate device, and the candidate device receives second biometric data corresponding to the bio signal measured by the patch-type bio signal measuring device for a second time based on information on the requested biometric data from the patch-type bio signal measuring device.

13. The biometric data management system of claim 11, wherein the smart watch selects the candidate device from a list of the candidate devices provided from the server.

14. The biometric data management system of claim 11, wherein the information on the requested biometric data includes a packet number.

15. The biometric data management system of claim 12, wherein the second biometric data is generated from a second bio signal subsequent to a first bio signal corresponding to the first biometric data.

16. The biometric data management system of claim 11, wherein when it is determined that the second residual amount of the battery is sufficient, wireless communication is re-connected between the patch-type bio signal measuring device and the smart watch, and after the re-connecting, the smart watch receives second biometric data corresponding to a bio signal measured by the patch-type bio signal measuring device for a second time from the patch-type bio signal measuring device.

17. The biometric data management system of claim 11, wherein the data transmission cycle is set,
the wireless communication is connected to the patch-type bio signal measuring device, and
the smart watch requests the patch-type bio signal measuring device to start measuring a bio signal.

18. The biometric data management system of claim 11, wherein the data transmission cycle is variably determined according to at least one of a type of the bio signal, a communication environment, and a battery status.

19. The biometric data management system of claim 11, wherein the server analyzes the received biometric data and checks whether there is missing biometric data.

20. A non-transitory computer readable recording medium storing a program for executing the biometric data management method, the method comprising:
measuring a bio signal of a user by a patch-type bio signal measuring device;
checking a first residual quantity of a battery of a smart watch;
when it is determined that the first residual amount of the battery is sufficient, connecting wireless communication between the patch-type bio signal measuring device and the smart watch, and receiving, by the smart watch, first biometric data corresponding to the bio signal measured by the patch-type bio signal measuring device for a first time from the patch-type bio signal measuring device;
transmitting, by the smart watch, the first biometric data received from the patch-type bio signal measuring device to a server;
disconnecting the wireless communication between the patch-type bio signal measuring device and the smart watch, and entering the smart watch into a standby mode for a predetermined data transmission cycle;
after the predetermined data transmission cycle, checking, by the smart watch, a second residual quantity of the battery; and
when it is determined that the second residual quantity of the battery is not sufficient, transmitting, by the smart watch or the server, information on requested biometric data to a candidate device.
